(19) United States
(12) Patent Application Publication
(10) Pub. No.: US 2015/0086979 A1
(43) Pub. Date: Mar. 26, 2015

(54) Method for Detecting Single Molecules in Living Cells and System for Use

(71) Applicant: Georg-August-Universitat Gottingen Stiftung Offenlichen Rechts, Gottingen (DE)

(72) Inventors: Christoph Schmidt, Friedland (DE); Nikita Fakhri, Goettingen (DE)

(73) Assignee: Georg-August-Universitat Gottingen Stiftung Offenlichen Rechts, Gottingen (DE)

(21) Appl. No.: 14/036,002

(22) Filed: Sep. 25, 2013

(51) Int. Cl. G01N 33/543

(52) U.S. Cl. CPC G01N 33/54313
USPC 435/6.1; 435/287.2; 536/23.1

(57) ABSTRACT

The present invention relates in a first aspect to a method for detecting single molecules in living cells based on tracking single molecules labelled with SWNTs (Single-Walled Carbon Nanotube). In a further aspect, the present invention provides a kit comprising at least a SWNT for use in time resolved determination of single molecules in living cells as well as to a system for detecting the presence, in particular, the trajectories of single cells in living cells.

The present invention relates in a first aspect to a method for detecting single molecules in living cells based on tracking single molecules labelled with SWNTs (Single-Walled Carbon Nanotube). In a further aspect, the present invention provides a kit comprising at least a SWNT for use in time resolved determination of single molecules in living cells as well as to a system for detecting the presence, in particular, the trajectories of single molecules in living cells.

### PRIOR ART

Trafficking of macromolecules and organelles is central to cell function. As for example, proteins produced in the endoplasmic reticulum are shuttled via specific cell compartments to their destination. Endocytosis and exocytosis also rely on intracellular transport. Outlying areas of cells, such as the extended axons of neurons, need to be supplied by directed transport. Intracellular traffic is driven mostly by kinesin and dynein motor proteins, carrying cargo along a radial network of microtubules (MTs). Kinesin motors have been studied in single-molecule experiments in vitro, but the dynamics of individual motors in living cells and whole organism remains much less explored. In the literature aspects of traffic regulation and addressing are discussed, but further progress is required to be able to follow the long-time and long-distance motion of biomacromolecules inside cells. To achieve this goal by commonly applied fluorescence microscopy, the following requirements must be met: (i) stable, slowly bleachable probes, (ii) high signal-to-noise ratio and (iii) efficient targeting of probes to specific molecules inside the cell.

Today optical equipment in conjunction with optimized fluorescent dyes can resolve and track single molecules with high temporal and spatial resolution in vitro, e.g. Kim, H. & Ha, T. Rep Prog Phys, 2013, 76, 016601. However, single molecule imaging in cells is hampered by serious obstacles. It has been done with short recording times or in limited cellular sub-volumes, such as the cell membrane. The temporal resolution and recording times are severely limited due to the lack of photostability of the dyes. In addition, with the present techniques, in particular, the signal-to-noise ratios tend to be marginal due to autofluorescence of other cellular components, which overlaps with typical fluorophore emission spectra. A technique described to mitigate the fluorescent background problem is Total-Internal-Reflection-Fluorescence microscopy which is able to reduce the background and makes it possible to image individual fluorescent molecules in the cell periphery, e.g. described in Schaaf, M. J. et al., Biophys. J., 2009, 97, 1206-1214. However, the limited optical penetration depth makes it impossible to image with this technique much beyond the cell membrane.

In WO 2009/042689 carbon nanotube compositions and methods for production thereof are described. These carbon nanotubes also called SWNT (single-walled carbon nanotube) are stiff 1-D tubular all-carbon nanostructures, with diameters around 1 nm and persistence lengths above 10 μm. Short nanotubes of up to hundreds of nanometer in length behave essentially as rigid rods. Individual semiconducting SWNTs luminesce with large Stokes shifts in the near infrared (˜900-1400 nm) depending on their chirality. In WO 2009/042689 the behaviour of such SWNTs is described. The identified luminescence is in the near infrared range, a window that is virtually free of autofluorescence in biological tissues. SWNT photoluminescence is excitonic. Fluorescence emission is highly stable with ultra-slow photobleaching and no blinking, allowing for long-term tracking. In addition, SWNTs can be introduced into cells and organisms without affecting viability.

In WO 2012/030961 a carbon nanotube array for optical detection of protein interactions is described. The composition described therein includes a nanostructure and a linker associate with a nanostructure, wherein the linker is configured to interact with a capture protein. The nanostructure can include the SWNT. A plurality of the configuration can be configured in an array.

As mentioned before, experiments aimed at following the long time, long distance motions of individual molecules in cells encounter serious obstacles. The present invention provides methods, kits and a system overcoming at least some of said obstacles.

### DESCRIPTION OF THE PRESENT INVENTION

In a first aspect, the present invention relates to a method for detecting single molecules in living cells, comprising the steps of
providing living cells containing single molecules having a first binding partner;
providing a Single-Walled Carbon Nanotube (SWNT) functionalized with a polymer having a second binding partner being configured for interaction with the first binding partner of the single molecules present in the living cells;
introducing said SWNT into said living cells containing single molecules having a first binding partner;
allowing the formation of a complex of the SWNT and the single molecule present in the cell through binding of the first to the second binding partner;
irradiating the living cells containing the SWNT with suitable wavelengths for exciting fluorescence of the SWNTs;
detecting the presence, in particular, the location of the single molecules based on the fluorescence emitted by the excited SWNT-single molecule complex in the infrared range.

That is, the present inventors recognized that the use of SWNTs for labelling single molecules in living cells allows to detect and determine the location of said single molecules whereby the use of SWNT as fluorophore allows to detect and track time-resolved distributions or trajectories of said single molecules by determining luminescence, such as fluorescence, in the infrared range.

That is, the use of the fluorescent SWNTs represents a unique tool for intracellular single-molecule tracking. This approach allows an efficient targeting being flexible due to the use of the binding partners. Furthermore, the SWNTs are not affecting viability and, after introduction into the cells, e.g. by electroporation or injection, find their targets promptly. That is, surprisingly, the functionalized SWNT bound via the binding partner to the single molecule does not affect the viability of the cells nor influence significantly the physiology of the cells. Moreover, due to the photostability of the SWNTs, the problem of photobleaching is not given and, in addition, tracking is not hindered by high fluorophore concentrations or blinking. Since the detection of the fluorescence signal is in the infrared range, the problem of autofluorescence is diminished. The signal-to-noise ratio in images of whole cells or even whole organisms is high, even under wide-field illumination.

Further, imaging of long-range transport as well as long time observations are possible, thus enabling to study long-distance trajectories of individual molecules in living cells. The method provides new approaches to observe and detect biomolecular dynamics of single molecules in the full physiological context of the living cell or organism. The biomolecular dynamics includes observation of interaction of biomacromolecules with high spatial and temporal complexity.

The use of SWNTs labels for single-molecule tracking of one or more molecular species breaks important barriers that have impeded such observations in the past.

As used herein, the term “detecting” includes imaging and tracking. Tracking identifies that the location of the observed molecule is determined at least twice, thus, enabling to visualise a movement of the same.

As used herein, the term “comprise” or “contain” includes the embodiments of “consist”.

As used herein, the term “functionalized” refers to embodiments wherein the polymer is covalently or non-covalently attached to the SWNT. The attachment may include a wrapping of the SWNT, e.g. as described in WO 2012/030961 or WO 2009/042689. In addition, “functionalized” refers to functionalization with polymers having the second binding partner.

As used herein, the term “antibody” includes all known types of antibodies, like single chain antibodies, antibody fragments etc. The antibodies bind specifically to the single molecules without impeding their moving or movement abilities.

The term “polymer” identifies structures containing repeating units of monomers. In particular, the polymer is a biopolymer. The term biopolymer identifies polymers produced by living organisms including polynucleotides, polypeptides as well as polysaccharides. The term “polymer” includes oligomers as well as polymers. That is, the term “polymers” including polynucleotides, polypeptides as well as polysaccharides refers also to oligonucleotides, oligopeptides as well as oligosaccharides whereby the oligonucleotides have at least 20 nucleic acid length, the oligopeptides have at least 10 amino acid length and the oligosaccharides include at least 10 monomer structures.

The term “infrared range” refers to the range of about 900 to 1400 nm wavelength.

The method as described herein includes in vivo as well as in vitro methods. In addition, the living cells can be isolated cells or may be part of a whole organism.

The method according to the present invention includes the step of introducing SWNTs into the living cells, whereby the living cells contain single molecules having a first binding partner. The SWNTs are functionalized with a polymer having a second binding partner, whereby said second binding partner is configured for interaction with the first binding partner of the single molecules present in the living cells.

The living cells provided in the method according to the present invention contain single molecules having a first binding partner. This first binding partner may be a first binding partner naturally formed by the single molecule as such. Alternatively, the first binding partner may be a molecule binding specifically the single molecule, like an antibody, e.g. a single chain antibody or fragments of an antibody. In addition, another embodiment of the single molecule having a first binding partner is a molecule composed of at least two components, e.g. a fusion protein containing the single molecule and the binding partner. Typical examples of binding partners include known protein tags. The skilled person is well aware of suitable protein tags usable in living cells.

In case where the single molecules are molecules composed of fused components, these single molecules are typically introduced into the cells by genetic engineering. For example, in case of fusion proteins composed of a protein or polypeptide to be detected in the cell and the first binding partner, said fusion proteins may be introduced by electroporation or transfection using appropriate means. For example, in case of transfection, appropriate vectors are introduced into cells accordingly.

It is preferred that the single molecules to be detected are recombinant molecules having a first binding partner obtained by genetic engineering. In this case, the first binding partner is preferably a protein tag known to the skilled person including a snap-tag or a halo-tag.

In another embodiment, the single molecule may be a molecule naturally occurring in the living cells and the first binding partner of said molecule is an antibody introduced into said living cells accordingly. In case of antibodies or other molecules as first binding partner of a molecule naturally occurring in the living cells or a molecule which had been introduced separately into the living cells, the first binding partner may be associated directly with the SWNTs. That is, the antibodies may be linked to the functionalized SWNTs as described herein.

The skilled person is well aware of suitable methods and means for genetically engineering said cells and for introducing appropriate first binding partners into said cells.

The SWNTs suitable according to present inventions are SWNTs functionalized with a polymer having a second binding partner. Said second binding partner is configured for interaction with the first binding partner of the single molecule present in the living cells. The first binding partner and the second binding partner are able to bind to each other, thus, forming a complex of the SWNT and the single molecule through binding of the first to the second binding partner.

The skilled person is well aware of suitable SWNTs useful according to the present invention. For example, SWNT which may be used according to the present invention are described in WO 2009/042689 and WO 2012/030961.

WO 2009/042689 and WO 2012/030961 are incorporated herein by reference in full.

The SWNT useful according to the present invention has preferably a predetermined diameter. Depending on the wavelengths to be determined in the near infrared range, the diameter of the SWNT is selected. Further, the SWNT according to the present invention have a length of at least 90 nm.

That is, in a preferred embodiment, the method according to the present invention is for detecting at least two different types (or more) of single molecules in the living cells wherein said single molecules having a first binding partner are different from each other and having different first binding partner; and providing distinguishable SWNTs whereby said SWNT are different in diameter, and have different second binding partner, the detection and measuring of the excitation of the irradiated SWNTs is effected at different wavelengths allowing resolution and differentiation of the distinguishable SWNTs emitting with different wavelengths, in particular, wherein the difference in the emission spectra of said distinguishable different SWNT is at least 20 nm, preferably, at least 50 nm.

In particular, the difference in the emission spectra of the distinguishable different SWNTs, whereby the SWNTs are different in diameter, allows to determine specifically signals of these distinguishable SWNT. For example, the SWNTs have differences in the emission spectra of at least 20 nm, preferably at least 50 nm, like at least 100 nm. In an embodiment of the present invention, the emission spectra of said distinguishable different SWNT are determined by different detectors. For example, while a first SWNT is determined with a first detector from a silicon based detector, a germanium-based detector, an indium-gallium-arsenide based detector, a platinum-silicide based detector, an indium-antimonide based detector, a mercury-cadmium-telluride based detector, the second or further SWNTs are detected the emission of the further or second SWNT is detected with a detector as identified above, but being different to the detector for the first SWNT. Of course, it is also possible that the same type of detector is used, e.g. the same type of detector equipped with different filters or determining the fluorescence at different areas of the same detector.

As mentioned before, it is within the scope of the method according to the present invention to detect at least two different types of single molecules. When detecting at least two different types of single molecules, said single molecules having a first binding partner are single molecules wherein the first binding partners are different from each other. For example, the single molecule A has a first binding partner B while the further single molecule C different from the first single molecule A, has a different first binding partner D. In addition, the distinguishable SWNTs, whereby said SWNTs are different in diameter, have different second binding partners. While the SWNT having a second binding partner E may form a complex with the first single molecule A having the first binding partner B, the distinguishable SWNT F having a different diameter than the first SWNT has a different second binding partner. The second SWNT F forms a complex with the second single molecule B having a first binding partner D.

Thus, it is possible to detect at least two different types of single molecules in a living cell at the same time allowing time resolved resolution and determination of trajectories of said single molecules. As identified before, the distinguishable SWNTs have different diameters, thus, have differences in the emission spectra allowing distinguishable detection of said SWNTs. For example, while the first single molecules having a first binding partner are single molecules coupled to a HIS-tag, the SWNT with the second binding partner are SWNTs being functionalized with polynucleotides having linked thereto NTA. The second single molecule having a first binding partner different to the first single molecule may have a Halo-tag while the SWNT has a Halo-ligand attached.

The SWNT is a functionalized SWNT. Functionalization of SWNTs is described for example in WO 2012/030961 which is incorporated herein by reference in full. In particular, the SWNT according to the present invention is functionalized with a polymer said polymer having a second binding partner configured for the interaction with the first binding partner of the single molecule.

The polymer used for functionalization of the SWNT is e.g. a biopolymer. Suitable biopolymers include polynucleotides, polysaccharides and polypeptides or an organic amphiphile. As used herein, the term polynucleotide includes nucleotides having a length of at least 20 nucleic acids or polypeptides having a length of at least 10 amino acids. In addition, the organic amphiphile useful according to the present invention include ionic or non-ionic surfactants including PEG, Pluronics, Sodium deoxycholate.

In a preferred embodiment, the polymer is a polynucleotide, in particular, an oligonucleotide. For example, the biopolymer is an oligonucleotide, like oligo d(T)30.

Suitable polymers are described in WO 2012/030961. Therein, suitable second binding partners are identified as well. For example, the polymer can be configured to act with the first binding partner by including a second binding partner in the polymer that can interact with the first binding partner. The second binding partner can include an ion. The skilled person is well aware of suitable ions. Further, the second binding partner may include a chelating region. A chelating region can include a chelator, which can be a polydendate ligand capable of forming two or more bonds, for example, the chelating region is the NTA region interacting with the His-tag ligand. NTA (Nα,Nα-bis(carboxymethyl)-L-lysine), linked to the amine group on SWNT-ssDNA via disuccinimidyl suberate, as known in the art.

Further, the first binding partner may be a protein tag, like a histidin tag, a chitin binding protein tag, maltose binding protein tag, glutathione-S-transferase tag, c-myc tag, FLAG-tag, V5-tag or HA-tag, SNAP or Halo-tag.

In an embodiment of the present invention, the method allows for detecting time-resolves distributions or trajectories of said single molecules in living cells whereby the step of detecting the presence, in particular, the location of the single molecules is performed at least twice to allow the determination of the spatial distribution of trajectories of the SWNT and, thus, the SWNT-single molecule complex, over the time.

For example, the location of the single molecules is determined over time with a time resolution between 1 ms and 1000 ms. That is, the method is conducted in real time. For example, a detection of the emission of SWNT is conducted every ms or every 10 ms. At least two detecting steps are performed for allowing time resolution, typically at least 10 detecting steps, e.g. at least 100, like at least 1000 detection steps (frames) are performed. For example, tracking is affected for at least one minute with at least 1000 frames, thus, allowing a long-distance long-time detection of the single molecule-SWNT complex. For example, the method is directed to a long-term observation recording at least 1000 frames, e.g. at least 10000 frames whereby the interval between two frames may vary between 1 ms up to 1000 ms. Hence, it is possible to determine the trajectories of these single molecules in living cells.

That is, the method according to the present invention allows to detect time-resolved distribution or trajectories of the single molecules in living cells without noticeably influencing the living cells. Hence, it is possible to track these single molecules in their native environment without impeding the cells.

In another embodiment, the single molecules with the first binding partner are cytoskeletal molecules or mechano-enzymes. For example, the single molecules are kinesins as cytoskeletal molecules, or ribosomes or receptors.

According to the present invention, the SWNTs may be introduced into the living cells by electroporation or injection.

Furthermore it is within the scope of the method according to the present invention that the SWNT and the recombinant single molecule having a first binding partner are introduced simultaneously into the living cell, for example, by injection or electroporation. Also, it is possible, that the SWNT functionalized with a polymer having a second binding partner and a vector, typically an expression vector, are introduced simultaneously into the living cell. Skilled persons are well aware of the suitable method for introducing the same into the cell as well as for culturing the living cells after introduction of the SWNTs and, optionally, the recombinant single molecule or a vector encoding the same.

In another aspect, the present invention relates to a kit comprising at least a SWNT being functionalized with a polymer with a second binding partner being configured for interaction with a first binding partner present in living cells; optionally living cells containing single molecules having a first binding partner or plasmids allowing expression of recombinant single molecules having a first binding partner or plasmids allowing expression of a first binding partner binding specifically to the single molecules to be detected for use in time resolved determination of said single molecules in living cells.

For example the kit is a kit comprising at least the SWNT being functionalized with a polymer as described herein and containing living cells containing single molecules having a first binding partner. Alternatively, said kit comprises the SWNTs as described therein together with plasmids allowing expression of recombinant single molecules having a first binding partner and/or plasmids allowing expression of a first binding partner binding specifically to the single molecules to be detected. For example, the plasmid may encode single chain antibodies binding specifically to the first binding partner without interfering with the natural function of said molecule.

In another aspect, a system for detecting the presence, preferably, the location or trajectories of single molecules in living cells comprising SWNT being functionalized with a polymer with a second binding partner being configured for interaction with a first binding partner present in living cells; optionally, a means for introducing said SWNT; a source for irradiating a first, and, optionally, a second wavelength and means allowing the detection of the presence and/or the localization of the fluorescence emitted by the SWNT.

The means for allowing the detection of the presence and/or the localization of the fluorescence emitted by the SWNT is selected from a silicon based detector, a germanium-based detector, an indium-gallium-arsenide based detector, a platinum-silicide based detector, an indium-antimonide based detector, a mercury-cadmium-telluride based detector.

For example, in case of detecting two different single molecules being different from each other using distinguishable SWNTs, the system has two different detectors allowing detection of the distinguishable SWNTs accordingly.

The kit or system according to the present invention may comprise additionally suitable means for culturing the living cells including cell media etc.

### FIGURES

FIG. 1 Labeling motors with SWNTs and in vitro motility assay. a, Schematic of kinesin molecular motor construct. The C-terminus of the motor is extended by a HaloTag (or in some experiments a His-tag). The HaloTag (His-tag) binds to the respective functional counterpart linked to the SWNT. b, Tracks of SWNT-labeled kinesins moving on a dense network of surface-attached microtubules in an in vitro motility assay in a maximum intensity projection image. Polarization of excitation light is marked by arrow (unpolarized detection). Red diamonds mark the beginning and end of the 2.5 min trajectory of a single motor. Fluorescence intensity was recorded between t₁ and t₂ (see d). c, Histogram of velocities of SWNT-labeled kinesins from b (N=15 different motor tracks). d, Intensity variation of the tracked SWNT fluorescence between t₁ and t₂ due to rotation of the SWNT with respect to the excitation polarization.

FIG. 2 SWNT-labeled kinesins in COS-7 cells. a, Tracks of SWNT-labeled kinesins in a COS-7 cell shown as 2D maximum-intensity projection. The nucleus is outlined with the dotted line. b, Centroid position of each SWNT. Tracks of many of the moving SWNTs show typical features of kinesin-driven motility, such as long and relatively straight unidirectional runs. Color-coding represents instantaneous velocities. c, Histogram of the scaling exponents of 2D-MSDs of motor trajectories (N=367 in 30 cells). d, Histogram of the magnitudes of velocity of SWNT-labeled kinesins, scored in 2 s segments along the trajectories.

FIG. 3 Analyzing single-molecule motor tracks in COS-7 cells. a, Top: Kymograph of a single SWNT-kinesin tracked over a distance of ˜40 μm showing stop-and-go movement, including brief reversal of direction (track marked by diamonds in FIG. 2a) Bottom: instantaneous velocity scored in time segments of 2 s. b, (Inset) 2D projection of the trajectory of a single kinesin first moving along microtubule tracks (filled symbols) and then detaching from the microtubule and moving randomly in a confined area (stationary phase or waiting state, open symbols). (Inset) Schematic of MSD decomposition into transverse and axial components with respect to MT. MSD during the moving state, decomposed into longitudinal (MSD_{II}) and transverse (MSD_{I}) movement with respect to the MT, compared to 2D-MSD of the motor during the stationary phase (open symbols). c, Histogram of MSD scaling exponent for motors in the stationary phase (N=50).

FIG. 4 Statistics of axial and transverse motor motions in COS-7 cells. a, Decomposition of a typical motor trajectory in the moving phase into longitudinal (left) and transverse components (right). b, MSD of longitudinal (MSD) and transverse (MSD_{I}) components of a run c, Histogram of MSD scaling exponents for longitudinal α) and transverse (α₁) components for motors in moving state (N=30 runs). (Inset) Schematic of a kinesin motor moving along a MT embedded in an actin-myosin network. d, Histogram of the timescale at which the transverse MSDs level off (N=30 runs).

FIG. 5 SWNT tagged kinesins in C. elegans neurons. a, Near-infrared fluorescence image of a C. elegans nematode 1 hour after injecting SWNTs close to the nerve ring. Neuronal somata are strongly stained and neuronal processes are clearly outlined, for example the ventral nerve cord along the left side of the worm. b, Maximum intensity projection of a neuronal process in the nerve cord. c, Maximum intensity projection of a cell body and axon of AWOL neuron. Two clusters of fluorescence in neuron soma (bottom left) are likely to be two Golgi stacks. d, Kymograph of the movement of a single motor along a neuronal process showing periods of movement in both directions, interspersed with pauses. e, Histogram of UNC-116 velocities in C. elegans neurons (N=13 runs in 3 worms)

The present invention will be described further by way of examples without limiting the present invention thereon.

### Examples

### Methods

DNA Wrapping of SWNTs:

1.0 mg HiPco SWNTs (batch number 189.2, Rice University) and 2 mg d(T)30 oligonucleotides (Zheng, M. et al. Nat Mater 2, 338-342 (2003)) with an amine-terminated group on the 5′ end (Invitrogen) were added to 2 ml DI water in a glass scintillation vial. The vial was placed on ice and sonicated (Vibra Cell, VC-50; Sonics and Materials) at a power of 10 W and 20 kHz for 90 min using a 2-mm diameter microprobe tip. After sonication, the sample was centrifuged at 16000 g for 90 min. The supernatant was carefully collected and filtered using a 4 ml Millipore Amicon ultracentrifugal filter device (MWCO 100 kDa). The SWNT-ssDNA samples were stored at 4° C.

SWNT-Halo Ligand:

50 mg HaloTag succinimidyl ester (O4) ligand (Promega) was dissolved in 50 μl of dry DMSO (Sigma) and added to 500 μl of 50 mg/L SWNT-ssDNA. The reaction was started by adding 60 μl PBS (10×; Invitrogen) at room temperature for 2 h. The excess succinimidyl ester was removed using an Amicon centrifugal filter (MWCO 100 kDa).

SWNT-BS₃-NTA:

In order to attach SWNTs to His-tag proteins, SWNT-BS₃-NTA conjugate was formed by crosslinking Nα,Nα-Bis(carboxymethyl)-L-lysine (NTA; Sigma) to the amine group on SWNT-ssDNA via Bis[sulfosuccinimidyl]suberate (BS₃; Thermo Scientific). The NTA-Ni²⁺ complex was formed by addition of excess Ni (II) chloride. Immediately before use, the BS₃ was dissolved in dry DMSO (Sigma) at 10-25 mM and added to the solution of SWNT-ssDNA and NTA-Ni²⁺ at a ssDNA:BS₃:NTA-Ni²⁺ ratio of 1:20-50:1. The reaction was quenched by addition of 1M Tris (pH 7.5) to a final concentration of 20 mM. SWNT-BS₃-NTA was incubated with 0.1 mg/ml of His-tag protein for 1 hour.

Plasmids:

All mammalian expression vectors were constructed by standard cloning procedures. PCR amplification was done using the Expand High Fidelity kit (Roche). Full-length human Kinesin-1 was amplified by PCR from the plasmid pENTR/D-Topo KIF5C digested with AsiSI and SacI and ligated into pFC14A HaloTag CMV Flexi vector (Promega), creating a Kinesin-1 fusion construct with a C-terminal HaloTag.

For worm expression, we employed a C. elegans modified expression vector pPB95.77 harboring a pan-neuronal promotor (rab-3) driving expression of kinesin-1 (UNC-116) fused to the C-terminal HaloTag. The gene encoded for the HaloTag protein without the stop codon was obtained by PCR from the HaloTag pHT2 vector (Promega) with a BglII site straddling the initial methionine codon and a PstI site attached to the 3′ end of unc-116. For monitoring expression level and distribution, a rab3::unc-116::gfp::halo-tag construct was used. For in vitro experiments, we used DK4mer (a chimera between a tetrameric kinesin-5 namely xenopous Eg5 and a kinesin-1 namely drosophila kinesin-1) and Nkin-433 (a dimeric kinesin-1 namely neurospora kinesin-1, truncated at aa 433) plasmid (Thiede, C. L., S.; et al., Biophys J 104, 432-441 (2013)).

C. elegans Strains and Generation of Transgenic Animals:

C. elegans strains were cultured at 20° C. on NGM plates. Wild-type N2 Bristol strain and unc-116 (e2310) was obtained from the C. elegans genetic center CGC (University of Minnesota).

Transgenic strains were generated by plasmid microinjection into the gonad at 10 ng/μl concentration. Functionality of the constructs was assessed by rescue of the unc phenotype. Crosses were performed using classical genetic approaches.

In Vitro Motility Assays:

Coverslips were plasma cleaned (PDC-002; Harrick Plasma, Ithaca, N.Y.) and silanized with 3-[2-(2-Aminoethylamino)ethyl-amino]propyl-trimethoxysilane (DETA; Sigma) for microtubule (MT) immobilization. Assay chambers were made from coverslips, microscope slides, and double-stick tape. Chambers were flushed with approximately three chamber volumes of motility assay mix (BRB80⁺) based on BRB80 buffer (80 mM PIPES/KOH, pH 6.8, 1 mM MgCl₂, 1 mM EGTA) containing 10 μM taxol (paclitaxel), 2 mM ATP, 4 mM MgCl₂, 10 mM DTT, 0.08 mg/ml catalase C40, 0.1 mg/ml glucose oxidase, and 10 mM glucose. MTs were polymerized and were attached to DETA coverslips with 5 min incubation, followed by 5 min incubation with 0.1 mg/ml casein in BRB80. Finally, SWNT-motor (motor: DK4mer or Nkin433) diluted in BRB80 was introduced in the chambers.

Cell Culture:

African green monkey kidney cells (COS-7; DSMZ ACC60) were cultured at 37° C. in a humidified atmosphere containing 5% CO₂ and grown continuously in Dulbecco's Modified Eagle's Medium (DMEM; Sigma) containing 1 mg/ml D-glucose and 4 mM L-glutamine supplemented with 10% FBS (Sigma) and 1% penicillin-streptomycin (Lonza). In a typical experiment, cells were plated in 75 cm² tissue culture flasks (Falcon or Sarstedt) at a concentration of 0.8-1×10⁶ cells/flask, grown for 2 days before transfection.

Transfection of Cells and Electroporation of SWNTs:

Transfection was performed using a 4D-Nucleofector (Amaxa Biosystems) by optimizing a protocol for COS-7 cells (solution SG, program FF120). In each nucleofection experiment, 1:1:1 ratio of Halo::KIF5C, pTagRFP-tubulin (Ex./Em.=555 nm/584 nm; Evrogen) and SWNT-Halo ligand was used.

The nucleofected cells were immediately transferred into fresh medium, let adhered to fibronectin-coated (Millipore) and plasma-cleaned glass or quartz coverslips and incubated for 24-72 h. For imaging, cells on the coverslips were sandwiched between two coverslips using layers of double stick tape and the chambers were sealed using VALAP (Waterman-Storer, C. M. Curr Protoc Cell Biol Chapter 13 (2001)).

Microinjection of SWNTs into C. elegans:

Microinjection needles (Femtotips II; Eppendorf) were loaded with 10 μl of SWNT solution. Nanotubes were injected close to the nerve ring in the head, in the gonad and along the ventral nerve cord. After injection, worms were transferred to NGM agar plates. After 1 hour, worms were immobilized on a coverslip using either agarose and polystyrene beads or Tetramisole hydrochloride (Sigma) in M9 buffer and then sandwiched between another quartz coverslip.

In some C. elegans experiments, Halo-SWNTs were injected into the distal arm of the gonad, which contains a central core of cytoplasm that is shared by many germ cell nuclei. Therefore, Halo-SWNT injected in the gonad was delivered to the progeny.

Experimental Setup:

(6,5) nanotubes in the sample (Ex./Em.=567/975 nm) were excited by a diode-pumped CW 561 nm laser (40 mW; Compass 561; Coherent Inc.), a high power CW 561 nm DPSS laser (500 mW; Cobolt Jive™; Cobolt) and a tunable Ti:Sapphire laser (Mira-900F; Coherent Inc.). A neutral density filter (NDC-50C-4M, Thorlabs) served to adjust the intensity of the beam. The beam diameter was expanded using two lenses with focal length f₁=40 mm and f₂=150 mm (Thorlabs). The beam was circularly polarized using a quarter-wave plate (AQWP05M-600; Thorlabs) and then focused into the back aperture of a high-NA objective (alpha Plan-Apochromat, 100×, NA=1.46; Zeiss). The fluorescence was collected with the same objective and passed through a dichroic beam splitter (630 DCXR; AHF Analysentechnik), further filtered using two filters: a 600 nm band pass filter (BP 630/75; Zeiss) for imaging RFP-microtubules or a 900 nm longpass filter (F47-900; AHF Analysentechnik) for imaging (6,5) nanotubes and focused onto a low-noise EMCCD camera (iX-on+DU-888; Andor Technology) using a tube lens (f_{T}=164.5 mm; Zeiss) or SWIR camera with InGaAs detector (XEVA-SHS-1.7-320 TE-1, Xenics). Images of SWNT dynamics were recorded at 2-200 frames per second. The emission spectrum of SWNTs was collected by a cryogenically cooled 1D InGaAs detector (OMA V; Roper Scientific) placed at the output of a spectrometer (Acton SP2150; Princeton Instruments).

Results

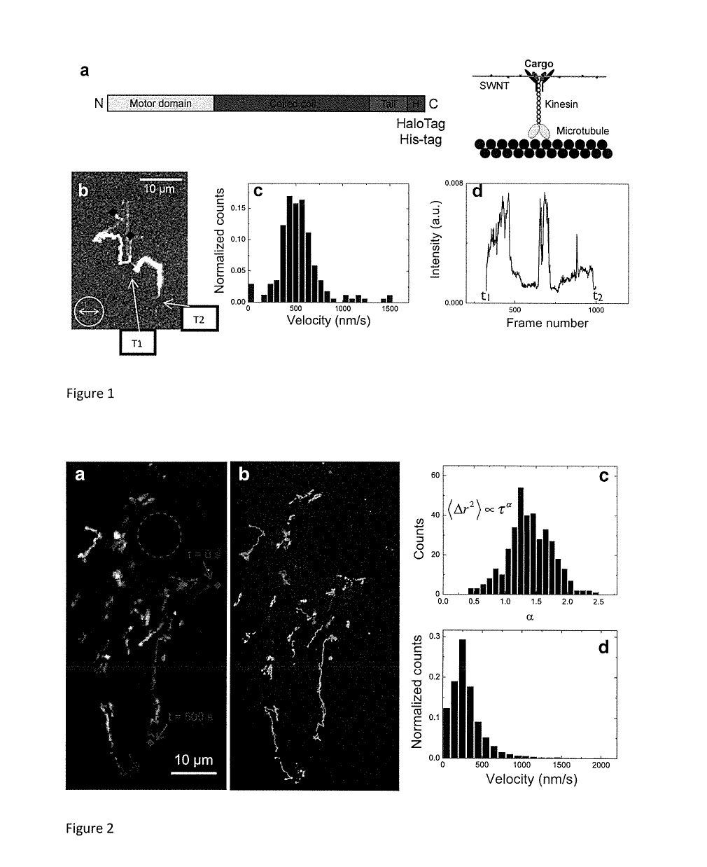

Here we specifically targeted SWNTs to kinesin motors to study intracellular transport, both in cultured COS-7 cells and in the neurons of C. elegans nematodes. We dispersed raw HiPco SWNTs in aqueous solutions by wrapping them with short DNA oligonucleotides (oligo(dT)30) with functional groups attached to the 5′ phosphate group. For in vitro studies, we used a kinesin expressed with a His-tag. For in vivo experiments, we utilized a crosslinking strategy based on a genetically engineered hydrolase (HaloTag) (Los, G. V. et al. ACS Chem Biol 3, 373-382 (2008)) as the mediator to covalently attach the nanotubes specifically to full-length kinesins, expressed in COS-7 cells and in the neuronal network of C. elegans (FIG. 1a).

To test the proper functionality of the motor, we labeled a tetrameric processive kinesin construct, consisting of kinesin-1 heads fused to a kinesin-5 stalk (Thiede, C. L., S.; et al., Biophys J 104, 432-441 (2013)), with SWNTs and observed motility in vitro. SWNT-labeled kinesins were detected in a custom-built near-infrared wide-field epifluorescence microscope, and sequences of images were recorded with a time resolution between 60 ms and 120 ms. FIG. 1 b shows the result of tracking single SWNT-labeled kinesins moving processively across a dense layer of substrate-adsorbed MTs at saturating ATP concentration. Velocities (443±113 nm/s, mean±S.D.) and run lengths (8±2 μm) are consistent with the known properties of this motor (FIG. 1c). The tracks of individual motors showed directed episodes interspersed with diffusive periods reflecting unbinding from or hopping between MTs. Fluorescence absorption and emission of SWNTs are strongly anisotropic and polarized along the nanotube axis. A rigidly attached SWNT therefore also reports on the orientation of the labeled protein. FIG. 1d shows the variation of fluorescence emission from a SWNT-labeled kinesin motor walking along MTs under linearly polarized excitation (polarization direction indicated in FIG. 1b). SWNTs were rigidly attached to the tetrameric kinesins in our experiments since we observed no variation in fluorescence intensity from SWNT-labeled kinesins that were bound tightly to MTs using the non-hydrolyzable ATP analog AMP-PNP in control experiments. Hence the variation of fluorescence intensity observed while the motors moved reflects changes in their orientation. While high intensity can only mean that the SWNT is oriented parallel to the excitation polarization, it was not possible to differentiate if a decrease in fluorescence intensity was caused by rotation in the focal plane or rotation out of the focal plane since fluorescence detection was not polarized. In the trace shown in FIGS. 1 b and d, for example, the first part of the vertical motor trajectory before t₂ clearly shows the SWNT at right angles to the MT axis, which is consistent with the SWNT bound parallel to the quadruple α-helix of the motor construct and the motor moving with one dimer engaged. Using circularly polarized excitation and simultaneous polarized detection along two axes will make it possible to unambiguously determine the SWNT orientation. Thus, the in vitro experiments confirmed that SWNTs performed as photostable, rigidly attached labels that did not interfere with the main motor functions.

To study the potential of nanotubes for in vivo single-molecule imaging, we labeled Kif5c, a kinesin-1 family member that functions as a cargo transporter in living cells. To achieve stable and specific attachment of SWNTs to a motor with native functions, full-length Kif5c was extended by a C-terminal HaloTag, a 34 kDa monomeric protein tag that cleaves carbon-halogen bonds in HaloTag ligands, halogenated aliphatic hydrocarbons. The HaloTag ligand was attached to DNA-wrapped SWNTs via crosslinking to the 5′ amine group on the oligonucleotide (see Methods). SWNTs with HaloTag ligands were introduced into Kif5c::Halo-expressing COS-7 fibroblasts by means of electroporation or microinjection in order to avoid trapping in endocytotic vesicles. The morphology and behavior of cells after microinjection or electroporation appeared normal.

The high photostability of SWNTs made it possible to only introduce a small number of them into cells (˜50-100 per cell) and still track individual SWNTs for long times. We used single-molecule tracking algorithms and determined the centroid position of each SWNT in the field of view to a precision of ˜20-50 nm. Fluorescent spots were detected with a signal-to-noise ratio of about 20 (integration time 60-500 ms). Intensity modulations reflect transient departures from the focal plane or SWNT rotation out of the focal plane, but not in the focal plane, since we used circularly polarized excitation.

Tracks of many of the moving SWNTs (FIGS. 2a and 2b) show typical features of kinesin-driven motility, such as long and relatively straight unidirectional runs. Dual-color imaging of SWNT-labeled kinesin and fluorescently-labeled MTs (pTagRFP-tubulin) demonstrated directly that SWNT-kinesin moved along MTs. Control experiments with cells not transfected with Kif5c::Halo showed essentially no linear long-distance movement of SWNTs. This observation confirms that directed trajectories are not caused by unbound SWNTs contained within vesicles that are transported by molecular motors.

To classify different observed modes of motion, we first analyzed trajectories by computing the mean-squared displacement (MSD), [symbol] Δr²(τ) [symbol] , which typically exhibits approximate power-law behavior [symbol] Δr²(τ) [symbol] ∝τ^{α}. Here, τ is the lag time and Δr(τ)=r(t+τ)−r(t) is the distance traveled in the focal plane in time τ. The power-law exponent α reflects the randomness of the motion, with α=0 for a stationary particle, α=1 for random diffusive motion and α=2 for regular directed motion on a straight track. We observed a broad distribution of exponents, α ranging between 0.5 and 2 (FIG. 2c). Some motors moved in straight lines with nearly constant velocity over the whole observation time (α≈2). The majority of trajectories, however, showed less directed motion with an exponent close to 1.4, an indication of mixed random/directed dynamics.

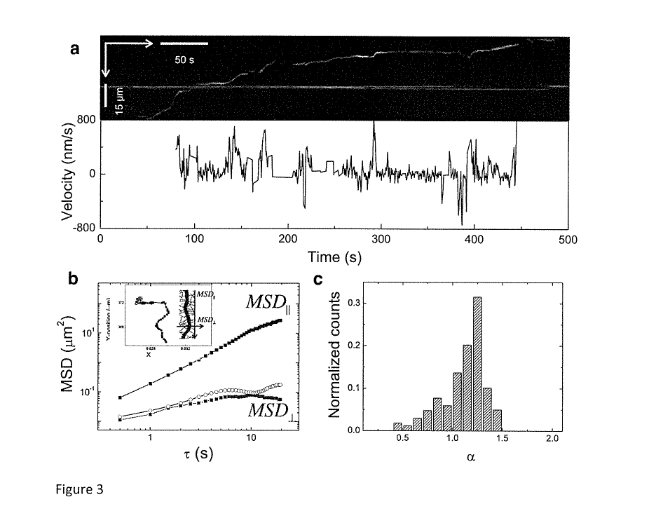

Tracking single motors with high accuracy throughout their intracellular travels reveals more detailed information about their dynamics. The velocities of straight runs, low-pass filtered over segments of 2 s were broadly distributed between 100 and 500 nm/s with an average of 300±7 nm/s (mean±standard error) (FIG. 2d). This confirms that the motion of kinesin is not significantly inhibited by the SWNT label, even in the crowded cellular environment. Several SWNT-kinesins could be tracked across the whole cell, over distances of tens of micrometers, much further than the average run length (˜1 μm) of kinesin-1 in vitro (FIG. 3a). Such extended runs are expected for cargo vesicles transported by more than one motor on a given MT, but could also be caused by a high density of MTs, with motors or cargo vesicles rapidly re-engaging on neighboring tracks after a release. Motors generally moved in a stop-and-go fashion along their tracks (FIG. 3a). Pauses might correspond to temporary detachment from the MT, but could also be caused by mechanical obstacles or regulatory interference. During phases of movement, kinesin velocity varied in magnitude and direction, predominantly pointing towards the cell periphery (FIG. 3a). Back-and-forth motion might have been due to switching to an oppositely oriented MT or due to dynein motors attached to the same cargo.

Cargo vesicles as well as MT tracks are embedded in the non-equilibrium viscoelastic cytoskeleton. Therefore the motion of tagged motors should reflect local fluctuations of the cytoskeleton in addition to motion relative to the MT. Consistent with this hypothesis, motors in stationary phases still moved, albeit randomly and in confined areas (FIG. 3b). The MSD analysis of such tracks showed scaling exponents, a, close to unity at short times (FIG. 3b), which, at first glance, suggests ordinary thermal diffusion in a purely viscous liquid. On closer inspection, however, these tracks exhibited a distribution of exponents (FIG. 3c) with a mean value≳1 (1.1±0.2) which is not possible for thermal diffusion in any medium, viscous or viscoelastic. Occasionally we observed transitions from the waiting state to the transport state (FIG. 3b inset), indicating that the stationary motors are not intrinsically different from the moving ones.

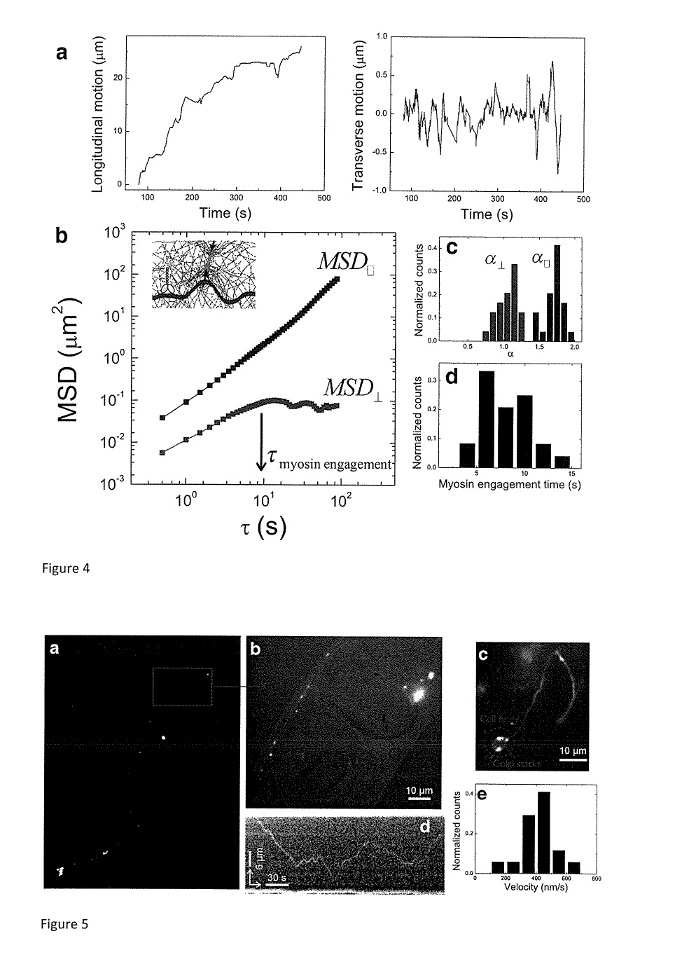

Interestingly, we see additional evidence for the hypothesis of active, motor-generated track fluctuations by analyzing the off-axis movement of motors traveling on MTs. The fact that MTs are rigid and therefore locally straight allows us to extract track fluctuations by decomposing trajectories into longitudinal (axial) and transverse (off-axis) components (FIG. 4a). The transverse component should mainly reflect the MT track dynamics, but would also include possible lateral switches to parallel tracks. The transverse MSD, indeed, showed approximately diffusive-like scaling α≈1) for short times (FIGS. 4b and 4c), leveling off for times ≳7 s (FIG. 4d). The time scale at which the MSDs leveled off is roughly equal to a typical cytoplasmic myosin motor-engagement time measured within cells.

The ultimate challenge for single-molecule fluorescence microscopy is dynamic imaging of single molecules in whole living organisms. Until now, single-molecule fluorescence studies in living systems came mostly from individual cells. Recently, single molecules were imaged in epidermal cells of zebrafish embryos using TIRF microscopy (Schaaf, M. J. et al. Biophys J 97, 1206-1214 (2009)). It will be extremely powerful to be able to track intracellular single-molecule dynamics in whole living organisms. We applied our approach to living Caenorhabditis elegans nematodes, an established model organism with a particularly well-charted neuronal network. C. elegans is well suited for microscopy, because the worms are small, transparent, and easy to manipulate. We generated transgenic lines of C. elegans expressing the kinesin-1 homolog unc-116 fused to a C-terminal HaloTag. The motor was expressed preferentially in neurons under the pan-neuronal rab-3 promotor. We microinjected SWNTs, functionalized with Halo ligands as described above, mostly close to the nerve ring near the head. Within 60 min after injection, we typically observed fluorescence in the whole animal down to the tail, but preferentially in the neuronal network (FIG. 5a). Neuronal somata were strongly stained, and neuronal processes were clearly outlined, for example along the ventral nerve cord. The fact that the SWNTs spread rapidly and selectively in neurons is evidence for coupling to UNC-116 and directed transport. Control experiments in wild-type worms showed no preference for neurons. Tagged proteins could also be detected in worm embryos at the 3-fold stage of development (FIG. 5b). This opens up the interesting possibility to follow proteins into the progeny.

Zooming in on individual axons (FIG. 5b) demonstrates that fluorescent background is again very low in spite of wide-field imaging in a whole animal, and that individual SWNTs can be imaged. The majority of fluorescent SWNTs were clustered and immobile along the axons for the observation time of 10-20 min. In some somata two clusters of fluorescence appeared, which are likely to mark the two Golgi stacks (FIG. 5c). Unlike vertebrate cells, which contain one large juxtanuclear Golgi stack, C. elegans neurons contain two to three Golgi ministacks scattered throughout the cell. Accumulations of immobile SWNTs in the somata and the axons or dendrites might therefore highlight motors attached to Golgi stacks in a waiting state. We also could observe movement of individual UNC-116::Halo-SWNTs along neuronal processes. Kymograph analysis (FIG. 5d) reveals periods of movement in both directions, interspersed with pauses. Bidirectional movement in axons would indicate that the tagged motors are attached to cargo together with other motors, likely including dynein. In dendrites, the directionality of MTs is mixed, so that motion towards and away from the cell body could be driven by UNC-116. Tracking the velocities, low-pass filtered over 2 s, (FIG. 5e) reveals a distribution centered at ˜450 nm/s with a width of 150 nm/s. This speed is consistent with typical kinesin-1 transport. Tracks mostly ended when the motor moved out of the focal plane. Tracking for longer times will require a feedback system, keeping an individual motor in focus. In summary, our observations demonstrate that specific targeting of SWNTs is feasible and efficient in living C. elegans worms and that the advantages provided by SWNT fluorescent properties carry over to single-molecule imaging in whole living animals.

Our results establish fluorescent SWNTs as uniquely appropriate for intracellular single-molecule tracking. Targeting is efficient and flexible using the HaloTag system. SWNTs promptly find their targets after electroporation or injection. Observation times are not limited by photobleaching, and tracking is not hampered by high fluorophore concentrations or blinking. The signal-to-noise ratio in images of whole cells or whole organisms is high, even under wide-field illumination. Our recordings of kinesin-1 motility show intriguing long-range transport dynamics along MTs, and are precise enough to even see effects of the non-equilibrium random motions of the tracks themselves in the cell. The method introduced here opens a new window on biomolecular dynamics in the full physiological context of the living cell or organism. The functions of a living cell involve interactions of biomacromolecules with high spatial and temporal complexity. One can only reach a full understanding of these functions if one can observe the interacting molecular players through the completion of dynamic cellular processes. The use of carbon nanotube labels for single-molecule tracking of one or more molecular species breaks important barriers that have impeded such observations in the past.

1. A method for detecting single molecules in living cells, comprising the steps of
providing living cells containing single molecules having a first binding partner;
providing a Single-Walled Carbon Nanotube (SWNT) functionalized with a polymer having a second binding partner being configured for interaction with the first binding partner of the single molecules present in the living cells;
introducing said SWNT into said living cells containing single molecules having a first binding partner;
allowing the formation of a complex of the SWNT and the single molecule present in the cell through binding of the first to the second binding partner;
irradiating the living cells containing the SWNT with suitable wavelengths for exciting the fluorescence of SWNTs;
detecting the presence, in particular, the location of the single molecules based on the fluorescence emitted by the excited SWNT-single molecule complex in the infrared range.

2. The method according to claim 1 for detecting time-resolved distribution or trajectories of said single molecules in living cells whereby the step of detecting the presence, in particular, the location of the single molecules is performed at least twice to allow the determination of the spatial distribution or trajectories of the SWNT over the time.

3. The method according to claim 1, wherein the living cells are part of a whole organism.

4. The method according to claim 1, wherein the living cells contain recombinant molecules having a first binding partner being obtained by genetic engineering and/or the first binding partner of said single molecule is an antibody.

5. The method according to claim 1, wherein the single molecules having the first binding partner are cytoskeletal molecules or mechano enzymes.

6. The method according to claim 1, wherein the first binding partner is a protein tag includes a SNAP-tag or a Halo-tag.

7. The method according to claim 1, wherein said SWNT have a predetermined diameter.

8. A method according to claim 1, wherein the polymer is a biopolymer, in particular, a polynucleotide, a polysaccharide, a polypeptide or an organic amphiphile.

9. The method according to claim 1, wherein the location of the single molecules is determined over time, in particular, with an interval of time resolution between 1 ms and 1000 ms with at least 1000 frames.

10. A method according to claim 1, wherein introduction of said SWNTs is conducted by electroporation.

11. A method according to claim 1 for detecting at least two different types of single molecules in the living cells wherein said single molecules having a first binding partner are different from each other and having different first binding partner; and providing distinguishable SWNTs whereby said SWNT are different in diameter, and have different second binding partner, the detection and measuring of the excitation of the irradiated SWNTs is effected at different wavelengths allowing resolution and differentiation of the distinguishable SWNT emitting with different wavelengths, in particular, wherein the difference in the emission spectra of said distinguishable different SWNT is at least 20 nm, preferably, at least 50 nm.

12. The method according to claim 1 for tracking said single molecules in living cells.

13. The method according to claim 12 whereby tracking of said single molecules is conducted in real time.

14. The method according to claim 12 whereby tracking is affected for at least 1 minute.

15. The method according to claim 4 wherein the genetic engineering of the cells with the recombinant molecules having a first binding partner and the introduction of SWNT is conducted simultaneously.

16. The method according to claim 1 wherein the SWNT is at least 90 nm in length.

17. A kit comprising at least a SWNT being functionalized with a polymer with a second binding partner being configured for interaction with a first binding partner present in living cells; optionally living cells containing single molecules having a first binding partner or plasmids allowing expression of recombinant single molecules having a first binding partner or plasmids allowing expression of a first binding partner binding specifically to the single molecules to be detected for use in time resolved determination of said single molecules in living cells.

18. A system for detecting the presence, preferably, the localization or trajectories of single molecules in living cells comprising SWNT being functionalized with a polymer with a second binding partner being configured for interaction with a first binding partner present in living cells; optionally, a means for introducing said SWNT; a source for irradiating a first, and, optionally, a second wavelength and means allowing the detection of the presence and/or the localization of the fluorescence emitted by the SWNT.

19. The system according to claim 18 wherein the means for allowing the detection of the presence and/or the localization of the fluorescence emitted by the SWNT is selected from a silicon based detector, a germanium-based detector, an indium-gallium-arsenide based detector, a platinum-silicide based detector, an indium-antimonide based detector, a mercury-cadmium-telluride based detector.
